(19) United States
(12) Patent Application Publication
(10) Pub. No.: US 2003/0228691 A1
(43) Pub. Date: Dec. 11, 2003

(54) Processes for inhibiting gene expression using polynucleotides

(76) Inventors: David L. Lewis, Madison, WI (US); David B. Rozema, Madison, WI (US); Jon A. Wolff, Madison, WI (US); James E. Hagstrom, Middleton, WI (US); Hans Herweijer, Madison, WI (US)

Correspondence Address: Mark K. Johnson, Mirus, 505 South Rosa Road, Madison, WI, 53719, US

(21) Appl. No.: 10/440,983

(22) Filed: May 19, 2003

Related U.S. Application Data
(60) Provisional application No. 60/381,514, filed on May 17, 2002.
(60) Provisional application No. 60/382,842, filed on May 23, 2002.

(51) Int. Cl. A61K 48/00

(52) U.S. Cl. 435/375; 514/44

(57) ABSTRACT

A process is provided for inhibition of specific gene expression in an animal cell by delivering a combination of RNA function inhibitors. Using a combination of inhibitors results in improved efficacy of gene expression inhibition. The process can be used to reduce gene expression in cells in vitro and in vivo.

### CROSS-REFERENCE TO RELATED APPLICATIONS

This application is related to prior provisional application Nos. 60/381,514 filed May 17, 2002 and 60/382,842 filed May 23, 2002.

### FIELD

The present invention generally relates to inhibiting gene expression. Specifically, it relates to inhibiting gene expression by delivery of a combination of a gene expression inhibitor and small interfering RNAs (siRNAs) to post-embryonic animals.

### BACKGROUND

Most genes function by expressing a protein via an intermediate, termed messenger RNA (mRNA) or sense RNA. The ability to specifically knock-down expression of a target gene by anti-mRNA agents has obvious benefits. For example, anti-mRNA agents could be used to generate animals that mimic true genetic “knockout” animals to study gene function. In addition, many diseases arise from the abnormal expression of a particular gene or group of genes. Anti-mRNA agents could be used to inhibit the expression of the genes and therefore alleviate symptoms of or cure the disease. For example, genes contributing to a cancerous state could be inhibited. In addition, viral genes as well as mutant genes causing dominant genetic diseases such as myotonic dystrophy could be inhibited. Inhibiting such genes as cyclooxygenase or cytokines could also treat inflammatory diseases such as arthritis. Nervous system disorders could also be treated. Examples of targeted organs would include the liver, pancreas, spleen, skin, brain, prostrate, heart etc.

RNA interference (RNAi) describes the phenomenon whereby the presence of double-stranded RNA (dsRNA) of sequence that is identical or highly similar to a target gene results in the degradation of messenger RNA (mRNA) transcribed from that target gene [Sharp 2001]. It has been found that RNAi in mammalian cells is mediated by short interfering RNAs (siRNAs) of approximately 21-25 nucleotides in length [Tuschl et al. 1999 and Elbashir et al. 2001]. The ability to specifically inhibit expression of a target gene by RNAi has obvious benefits. For example, RNAi could be used to study gene function. In addition, RNAi could be used to inhibit the expression of deleterious genes and therefore alleviate symptoms of or cure disease. SiRNA delivery may also aid in drug discovery and target validation in pharmaceutical research.

The use of antisense nucleic acid is another method whereby mRNA function is inhibited. Unlike RNAi mechanisms, antisense nucleic acids do not act through the dsRNA-induced silencing complex (RISC). The antisense polynucleotide interferes with mRNA function by base pairing with the mRNA. Antisense polynucleotides may be DNA, RNA, or nucleic acid analogs, such a morpholinos and peptide nucleic acids.

Methods that utilize gene expression inhibitor or oligonucleotide analogs or siRNAs alone result in, at best, 90-95% inhibition of gene expression. This level of inhibition is often not sufficient to give a mutant phenotype which is necessary to determine the function of the inhibited gene under normal or diseased states. We describe the delivery of a combination of an anti-mRNA agents in order to achieve high levels of gene inhibition.

The delivery of genetic material has a number of useful purposes. Delivery of genes to cells both in vivo and in vitro facilitates the study of gene function. Similarly, delivery of compounds, such as antisense polynucleotides and siRNA, which inhibit gene expression can also be used to study gene function. Inhibition of gene expression is useful both for basic research as well as pharmaceutical drug development.

The delivery of genetic material as a therapeutic, gene therapy, promises to be a revolutionary advance in the treatment of disease. Although, the initial motivation for gene therapy was the treatment of genetic disorders, it is becoming increasingly apparent that gene therapy will be useful for the treatment of a broad range of acquired diseases such as cancer, infectious disorders (AIDS), heart disease, arthritis, and neurodegenerative disorders (Parkinson's and Alzheimer's). Not only can functional genes be delivered to repair a genetic deficiency, but nucleic acid can also be delivered to inhibit gene expression to provide a therapeutic effect. Inhibition of gene expression can be affected by antisense polynucleotides, siRNA mediated RNA interference and ribozymes. Transfer methods currently being explored included viral vectors and physical-chemical methods.

A variety of methods and routes of administration have been developed to deliver pharmaceuticals that include small molecular drugs and biologically active compounds such as peptides, hormones, proteins, and enzymes to their site of action. Parenteral routes of administration include intravascular (intravenous, intra-arterial), intramuscular, intraparenchymal, intradermal, subdermal, subcutaneous, intratumor, intraperitoneal, and intralymphatic injections that use a syringe and a needle or catheter. The blood circulatory system provides systemic spread of the pharmaceutical. Polyethylene glycol and other hydrophilic polymers have provided protection of the pharmaceutical in the blood stream by preventing its interaction with blood components and to increase the circulatory time of the pharmaceutical by preventing opsonization, phagocytosis and uptake by the reticuloendothelial system. For example, the enzyme adenosine deaminase has been covalently modified with polyethylene glycol to increase the circulatory time and persistence of this enzyme in the treatment of patients with adenosine deaminase deficiency.

Transdermal routes of administration include oral, nasal, respiratory, and vaginal administration. These routes have attracted particular interest for the delivery of peptides, proteins, hormones, and cytokines, which are typically administered by parenteral routes using needles.

### SUMMARY

In a preferred embodiment, we describe a process for efficiently inhibiting gene expression in an animal cell comprising: delivering a combination of two or more RNA function inhibitors (hereafter referred to as inhibitors) together or sequentially to the cell. The inhibitors comprise sequence that is identical, nearly identical, or complementary to the same, different, or overlapping segments of a target gene sequence(s). A preferred combination comprises one inhibitor that is an siRNA and another inhibitor that is selected from the group consisting of antisense nucleic acid and ribozyme. A preferred antisense polynucleotide is a phosphorodiamidate morpholino oligonucleotides, (PMOs or morpholinos), peptide nucleic acids (PNAs) or a 2′-O-methyl oligonucleotide. The inhibitor may be formed outside the cell and then delivered to the cell. Alternatively, the inhibitor may be transcribed within the cell from of a DNA that is delivered to the cell. Delivery of a combination of inhibitors provides more efficient inhibition of gene expression than delivery of either inhibitor alone. The inhibitors may be delivered to cells in vivo, ex vivo, in situ, or in vitro. The cell can be an animal cell that is maintained in tissue culture such as cell lines that are immortalized or transformed. The cell can be a primary or secondary cell which means that the cell has been maintained in culture for a relatively short time after being obtained from an animal. The cell can also be a mammalian cell that is within a tissue in situ or in vivo.

In a preferred embodiment, we describe an in vivo process for delivering a combination of two or more inhibitors to a nonvascular cell in a mammal for the purposes of inhibition of gene expression comprising: making the inhibitors, injecting the inhibitors into a vessel, and delivering the inhibitors to a cell within a tissue thereby inhibiting expression of a target gene in the cell. Delivering the inhibitors to a nonvascular cell within a tissue comprises: increasing the pressure within the vessel by injecting a sufficient volume of fluid into the vessel, injecting the solution at a sufficient rate and occluding the flow of fluid away of the target tissue. The volume consists of an inhibitor in a pharmaceutically acceptable solution wherein the solution may contain a compound or compounds which may or may not complex with the inhibitor and aid in delivery. The inhibitors comprise sequence that is identical, nearly identical, or complementary to the same, different, or overlapping segments of a target gene sequence(s). A preferred combination comprises one inhibitor that is an siRNA and another inhibitor that is selected from the group consisting of antisense nucleic acid and nbozyme. A preferred antisense polynucleotide is a morpholino, PNA or a 2′-O-methyl oligonucleotide. The inhibitor may be formed outside the cell and then delivered to the cell. Alternatively, the inhibitor may be transcribed within the cell from of a DNA that is delivered to the cell. Delivery of a combination of inhibitors provides more efficient inhibition of gene expression than delivery of either inhibitor alone.

In a preferred embodiment, a process is described for increasing the transit of the inhibitors out of a vessel and into the cells of the surrounding tissue comprising: rapidly injecting a sufficient volume of solution containing the inhibitors into a vessel supplying the target tissue, thus forcing fluid out of the vasculature into the extravascular space. The process may further comprise constricting the flow of fluid into and/or out of the tissue and adding a molecule that increases the permeability of a vessel. The target tissue comprises the cells supplied by the vessel distal to the point of injection. For injection into arteries, the target tissue is the cells that the arteries supply with blood. For injection into veins, the target tissue is the cells from which the vein drains blood.

In a preferred embodiment, we describe an in vivo process for delivering the inhibitors to a mammalian cells consisting of: inserting the inhibitors into a vessel and applying pressure to the vessel proximal to the point of injection and target tissue. The process includes impeding fluid flow into and away from the target tissue through afferent and efferent vessels of the tissue by externally applying pressure to the vessels such as by compressing mammalian skin. Compressing mammalian skin includes applying a cuff over the skin, such as a sphygmomanometer or a tourniquet. Fluid flow through vessels may also be constricted by directly clamping the vessels such as by a clamp or a balloon catheter. The vessels are occluded for a period of time necessary to delivery the inhibitor without causing ischemic damage to the tissue.

In a preferred embodiment, the inhibitors may be delivered to a cell in a mammal for the purposes of inhibiting a target gene to provide a therapeutic effect. The target gene is selected from the group that comprises: dysfunctional endogenous genes and infectious agent genes. Dysfunctional endogenous genes include dominant genes which cause disease and cancer genes. The inhibitors may also be delivered to a mammalian cell in vivo for the treatment of a disease or infection. The inhibitors may reduce expression of a viral or bacterial gene. The inhibitors may reduce or block microbe production, virulence, or both. Delivery of the inhibitors may delay progression of disease until endogenous immune protection can be acquired. Viral genes involved in transcription, replication, virion assembly, immature viral membrane formation, extracellular enveloped virus formation, early genes, intermediate genes, late genes, and virulence genes may be targeted. Cellularly transcribed genes involved in bacterial pathogenicity may be targeted. Alternatively, the inhibitors may decrease expression of an endogenous host gene to reduce virulence of a pathogen. The inhibitors may be delivered to a cell in a mammal to reduce expression of a cellular receptor. For example, the lethality of Anthrax is primarily mediated by a secreted tripartite toxin which requires the mammalian anthrax toxin receptor (ATR) for cellular entry. Reducing expression of ATR may decrease Anthrax toxicity. Receptors to which pathogens bind may also be targeted.

In a preferred embodiment, the inhibitors are delivered to a mammalian cell for the purpose of facilitating pharmaceutical drug discovery or target validation or for research purposes. The mammalian cell may be in vitro or in vivo. Specific inhibition of a target gene can aid in determining whether inhibition of a protein or gene has a significant phenotypic effect. Specific inhibition of a target gene can also be used to study the target gene's effect on the cell.

Further objects, features, and advantages of the invention will be apparent from the following detailed description when taken in conjunction with the accompanying drawings.

### BRIEF DESCRIPTION OF THE FIGURES

FIGS. 1A-1D. A) Delivery of siRNA-Luc+. Maximal inhibition is achieved at 10 nM siRNA-Luc+. B) Delivery of morpholino-Luc+. Maximal specific inhibition is achieved at 100 nM morpholino-Luc+. C) Comparison of inhibitory power of siRNA-Luc+ (1.0 nM) alone, morpholino-Luc+ (100 nM) alone and siRNA-Luc+ (1.0 nM) plus morpholino (100 nM) together. When siRNA and morpholino are added together at these concentrations, the degree of inhibition is greater than either siRNA or morphlino alone. D) Comparison of inhibitory power of siRNA-Luc+ (10 nM) alone, morpholino-Luc+ (100 nM) alone and siRNA-Luc+ (10 nM) plus morpholino (100 nM) together. When siRNA and morpholino are added together at these concentrations, the degree of inhibition is greater than either siRNA or morphlino alone.

### DETAILED DESCRIPTION

The disclosed invention provides a method to inhibit target gene expression in a cell by delivering to the cell a combination of two or more RNA function inhibitors. We show that delivery of an siRNA results a maximum inhibition of gene expression of around 90%. Delivery of another type of inhibitor, an antisense morpholino, results in maximal inhibition of gene expression of around 85%. For some therapeutic and research purposes, 85-90% inhibition of gene expression may be insufficient to provide a desired effect. We now show that delivery of a combination of inhibitors, such as an siRNA together with an antisense nucleic acid, can inhibition gene expression up to 98.6%. Furthermore, the method provides increased inhibition of gene expression in cells both in vitro and in vivo.

Inhibitor may be formed outside the cell and then delivered or may be formed in the cell by transcription of a gene that is delivered to the cell. The cell with the target gene may be derived from or contained in any organism (e.g., plant, animal, protozoan, virus, bacterium, or fungus). Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

Several aspects of current pharmaceutical and biological research and therapeutic treatment are candidates for the described combination technology. For the purposes of target validation, gene inactivation allows the investigator to assess the potential therapeutic effect of inhibiting a specific gene product. Expression arrays can be used to determine the responsive effect of inhibition on the expression of genes other than the targeted gene or pathway. Other methods of gene inactivation, generation of mutant cell lines or knockout mice suffer from serious deficiencies including embryonic lethality, expense, and inflexibility. Also, these methods frequently do not adequately model larger animals. Development of a more robust and easily applicable gene inactivation technology that can be utilized in both in vitro and in vivo models would greatly expedite the drug discovery process.

The term deliver means that the inhibitor becomes associated with the cell thereby altering the properties of the cell by inhibiting function of an RNA. The inhibitor can be on the membrane of the cell or inside the cytoplasm, nucleus, or other organelle of the cell. Other terms sometimes used interchangeably with deliver include transfect, transfer, or transform. In vivo delivery of an inhibitor means to transfer the inhibitor from a container outside a mammal to near or within the outer cell membrane of a cell in the mammal. The inhibitor can interfere with RNA function in either the nucleus or cytoplasm.

A delivery system is the means by which a biologically active compound becomes delivered. That is all compounds, including the biologically active compound itself, that are required for delivery and all procedures required for delivery including the form (such volume and phase (solid, liquid, or gas) and method of administration.

A variety of methods are available for delivering an inhibitor to a cell. The process of delivering a nucleic acid to a cell has been commonly termed transfection or the process of transfecting and has also been termed transformation. The term transfecting as used herein refers to the introduction of foreign nucleic acid or other biologically active compound into cells. The biologically active compound could be used for research purposes or to produce a change in a cell that can be therapeutic. The delivery of nucleic acid for therapeutic purposes is commonly called gene therapy. The delivery of nucleic acid can lead to modification of the genetic material present in the target cell. The term stable transfection or stably transfected generally refers to the introduction and integration of exogenous nucleic acid into the genome of the transfected cell. The term stable transfectant refers to a cell which has stably integrated foreign nucleic acid into the genomic DNA. Stable transfection can also be obtained by using episomal vectors that are replicated during the eukaryotic cell division (e.g., plasmid DNA vectors containing a papilloma virus origin of replication, artificial chromosomes). The term transient transfection or transiently transfected refers to the introduction of foreign nucleic acid into a cell where the foreign nucleic acid does not integrate into the genome of the transfected cell. The foreign nucleic acid persists in the nucleus of the transfected cell. The foreign nucleic acid is subject to the regulatory controls that govern the expression of endogenous genes in the chromosomes. The term transient transfectant refers to a cell which has taken up foreign nucleic acid but has not integrated this nucleic acid.

A transfection reagent or delivery vehicle is a compound or compounds that bind(s) to or complex(es) with an inhibitor and mediates its entry into cells. Examples of transfection reagents include, but are not limited to, non-vrial vectors, cationic liposomes and lipids, polyamines, calcium phosphate precipitates, histone proteins, polyethylenimine, and polylysine complexes. A non-viral vector is defined as a vector that is not assembled within an eukaryotic cell including protein and polymer complexes (polyplexes), lipids and liposomes (lipoplexes), combinations of polymers and lipids (lipopolyplexes), and multilayered and recharged particles. It has been shown that cationic proteins like histones and protamines, or synthetic polymers like polylysine, polyarginine, polyomithine, DEAE dextran, polybrene, and polyethylenimine may be effective intracellular delivery agents. Typically, the transfection reagent has a component with a net positive charge that binds to the oligonucleotide's or polynucleotide's negative charge. The transfection reagent mediates binding of oligonucleotides and polynucleotides to cells via its positive charge (that binds to the cell membrane's negative charge) or via ligands that bind to receptors in the cell. For example, cationic liposomes or polylysine complexes have net positive charges that enable them to bind to DNA or RNA.

An RNA function inhibitor (“inhibitor”) comprises any nucleic acid or nucleic acid analog comprising a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. Inhibitors are selected from the group comprising: siRNA, microRNA or other nucleic acid that induces RNA interference (RNAi), dsRNA, RNA Polymerase III transcribed DNAs, ribozymes, and antisense nucleic acid, which may be RNA, DNA, or artificial nucleic acid. SiRNA comprises a double stranded structure typically containing 15-50 base pairs and preferably 19-25 base pairs and having a nucleotide sequence identical or nearly identical to an expressed target gene or RNA within the cell. Antisense polynucleotides include, but are not limited to: morpholinos, 2′-O-methyl polynucleotides, peptide nucleic acids (PNAs), DNA, RNA, polynucleotide analogs and the like. RNA polymerase III transcribed DNAs contain promoters, such as the U6 promoter. These DNAs can be transcribed to produce small hairpin RNAs in the cell that can function as siRNA ⁱ or linear RNAs that can function as antisense RNA. The inhibitor may be polymerized in vitro, recombinant RNA, contain chimeric sequences, or derivatives of these groups. The inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

An inhibitor can be delivered to a cell in order to produce a cellular change that is therapeutic. The delivery of an inhibitor or other genetic material for therapeutic purposes (the art of improving health in an animal including treatment or prevention of disease) is called gene therapy. The inhibitor can be delivered either directly to the organism in situ or indirectly by transfer to a cell ex vivo that is then transplanted into the organism. Entry into the cell is required for the inhibitor to block the production of a protein or to decrease the amount of a target RNA. Diseases, such as autosomal dominant muscular dystrophies, which are caused by dominant mutant genes, are examples of candidates for treatment with therapeutic inhibitors such as siRNA. Delivery of the inhibitor would block production of the dominant protein without affecting the normal protein thereby lessening the disease.

The term polynucleotide, or nucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A polynucleotide can be delivered to a cell to express an exogenous nucleotide sequence, to inhibit, eliminate, or alter expression of an endogenous nucleotide sequence, or to affect a specific physiological characteristic not naturally associated with the cell. Polynucleotides may contain an expression cassette coded to express an RNA. An expression cassette refers to a natural or recombinantly produced polynucleotide that is capable of expressing a gene(s). The term recombinant as used herein refers to a polynucleotide molecule that is comprised of segments of polynucleotide joined together by means of molecular biological techniques. The cassette contains the region of the gene of interest along with any other sequences that affect expression of the gene.

The polynucleotide may contain sequences that do not serve a specific function in the target cell but are used in the generation of the polynucleotide. Such sequences include, but are not limited to, sequences required for replication or selection of the polynucleotide in a host organism.

The term expression cassette refers to a natural or recombinantly produced nucleic acid molecule that is capable of expressing a gene. As used herein, the term “gene expression” refers to the process of converting genetic information encoded in a gene into RNA through “transcription” of a deoxyribonucleic gene (e.g., via the enzymatic action of an RNA polymerase). The term “gene” can refer to a nucleic acid sequence that comprises coding sequences necessary for the production of a therapeutic nucleic acid. The term encompasses the coding region of a gene as well as sequences located adjacent to the coding region on both the 5′ and 3′ ends which may affect expression of the gene.

According to the invention, the inhibitors can be delivered to cells in culture, i.e., in vitro. These include a number of cell lines that can be obtained from American Type Culture Collection (Bethesda) such as, but not limited to: 3T3 (mouse fibroblast) cells, Rat1 (rat fibroblast) cells, CHO (Chinese hamster ovary) cells, CV-1 (monkey kidney) cells, COS (monkey kidney) cells, 293 (human embryonic kidney) cells, HeLa (human cervical carcinoma) cells, HepG2 (human hepatocytes) cells, Sf9 (insect ovarian epithelial) cells and the like.

The invention further provides for the delivery of inhibitors to a cell that is in situ, ex vivo or a primary cell. Primary cells include, but are not limited to, primary liver cells and primary muscle cells and the like. For primary cells, the cells within the tissue are separated by mincing and digestion with enzymes such as trypsin or collagenases which destroy the extracellular matrix. Tissues consist of several different cell types. Purification methods such as gradient centrifugation or antibody sorting can be used to obtain purified amounts of the preferred cell type. For example, primary myoblasts are separated from contaminating fibroblasts using Percoll (Sigma) gradient centrifugation.

Parenchymal cells are the distinguishing cells of a gland or organ contained in and supported by the connective tissue framework. The parenchymal cells typically perform a function that is unique to the particular organ. The term “parenchymal” often excludes cells that are common to many organs and tissues such as fibroblasts and endothelial cells within blood vessels.

For example, in a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and opposed sides to a bile canaliculus. Liver cells that are not parenchymal cells include cells within the blood vessels such as the endothelial cells or fibroblast cells. In one preferred embodiment hepatocytes are targeted by injecting the inhibitor or inhibitor complex into the portal vein or bile duct of a mammal.

In striated muscle, the parenchymal cells include myoblasts, satellite cells, myotubules, and myofibers. In cardiac muscle, the parenchymal cells include the myocardium also known as cardiac muscle fibers or cardiac muscle cells and the cells of the impulse connecting system such as those that constitute the sinoatrial node, atrioventricular node, and atrioventricular bundle.

In addition, the invention provides a delivery system for the delivery of a combination of two or more inhibitors to cells in vivo. We have found that an intravascular route of administration allows an RNA function inhibitor (inhibitor) to be delivered to mammalian cells in a more even distribution than direct parenchymal injections. The efficiency of inhibitor delivery may be increased by increasing the permeability of the tissue's blood vessel. Permeability is increased by increasing the intravascular hydrodynamic pressure (above, for example, the resting diastolic blood pressure in a blood vessel), delivering the injection fluid rapidly (injecting the injection fluid rapidly), using a large injection volume, and/or increasing permeability of the vessel wall.

A needle or catheter is used to inject a solution containing the inhibitors or inhibitor-containing-complex into a vessel. A catheter can be inserted at a distant site and threaded through the lumen of a vessel so that it resides in a vascular system that connects with a target tissue. The injection can also be performed using a needle that traverses the skin and enters the lumen of a vessel.

Efficiency of inhibitor delivery is increased by increasing the permeability of a vessel and vascular system within the target tissue. Permeability is defined here as the propensity for macromolecules such as an inhibitor to exit the vessel and enter extravascular space. One measure of permeability is the rate at which macromolecules move out of the vessel. Another measure of permeability is the lack of force that resists the movement of inhibitors being delivered to leave the intravascular space.

Inserting into a vessel an appropriate volume at an appropriate rate increases permeability of the vessel to the injection solution and the molecules or complexes therein. Permeability can be further increased by occluding outflow of fluid (both bodily fluid and injection solution) from the tissue or local vascular network. For example, a solution is rapidly injected into an afferent vessel supplying an organ while the efferent vessel(s) draining the tissue is transiently occluded. Branching vessels may also be occluded. Natural occlusions may also be used. The afferent vessel into which the solution is inserted may also be transiently occluded proximal to the injection site. The vessels are partially or totally occluded for a period of time sufficient to allow delivery of a molecule or complex present in the injection solution. The occlusion may be released immediately after injection or may be released only after a determined length of time which does not result in tissue damage due to ischemia. The solution may also be inserted into an efferent vessel.

The permeability of a vessel may also be increased by increasing the osmotic pressure within the vessel. Typically, hypertonic solutions containing salts such as NaCl, sugars or polyols such as mannitol are used. Hypertonic means that the osmolarity of the injection solution is greater than physiological osmolarity. Isotonic means that the osmolarity of the injection solution is the same as the physiological osmolarity (the tonicity or osmotic pressure of the solution is similar to that of blood). Hypertonic solutions have increased tonicity and osmotic pressure relative to the osmotic pressure of blood and cause cells to shrink.

The permeability of a vessel can also be increased by a biologically-active molecule. A biologically-active molecule is a protein or a simple chemical such as papaverine or histamine that increases the permeability of the vessel by causing a change in function, activity, or shape of cells within the vessel wall such as the endothelial or smooth muscle cells. Typically, biologically-active molecules interact with a specific receptor or enzyme or protein within the vascular cell to change the vessel's permeability. Examples of drugs or chemicals that may be used to increase vessel permeability include histamine, vascular permeability factor (VPF, which is also known as vascular endothelial growth factor, VEGF), calcium channel blockers (e.g., verapamil, nicardipine, diltiazem), beta-blockers (e.g., lisinopril), phorbol esters (e.g., PKC), ethylenediaminetetraacetic acid (EDTA), adenosine, papaverine, atropine, and nifedipine. Another type of biologically-active molecule can increase permeability by changing the extracellular connective material. For example, an enzyme could digest the extracellular material and increase the number and size of the holes of the connective material.

The choice of injection volume and rate are dependent upon: the size of the animal, the size of the vessel into with the solution is injected, the size and or volume of the target tissue, the bed volume of the target tissue vasculature, and the nature of the target tissue or vessels supplying the target tissue. For example, delivery to liver may require less volume because of the porous nature of the liver vasculature. The precise volume and rate of injection into a particular vessel, for delivery to a particular target tissue, may be determined empirically. Larger injection volumes and/or higher injection rates are typically required for a larger vessels, target sizes, etc. For example, efficient delivery to mouse liver may require injection of as little as 1 ml or less (animal weight ˜25 g). In comparison, efficient delivery to dog or nonhuman primate limb muscle may require as much as 60-500 ml or more (animal weight 3-14 kg). Injection rates can vary from 0.5 ml/sec or lower to 4 ml/sec or higher, depending on animal size, vessel size, etc. Occlusion of vessels, by balloon catheters, clamps, cuffs, natural occlusion, etc, can limit or define the vascular network size or target area.

The injection volume can also be related to the target tissue. For example, delivery of a non-viral vector with an inhibitor to a limb can be aided by injecting a volume greater than 5 ml per rat limb or greater than 70 ml for a primate limb. The injection volumes in terms of ml/limb muscle are usually within the range of 0.6 to 1.8 ml/g of muscle but can be greater. In another example, delivery of an inhibitor or inhibitor complex to liver in mice can be aided by injecting the inhibitor in an injection volume from 0.6 to 1.8 ml/g of liver or greater. In another example delivering an inhibitor to a limb of a primate (rhesus monkey), the inhibitor or complex can be in an injection volume from 0.6 to 1.8 ml/g of limb muscle or anywhere within this range.

In another embodiment the injection fluid is injected into a vessel rapidly. The speed of the injection is partially dependent on the volume to be injected, the size of the vessel into which the volume is injected, and the size of the animal. In one embodiment the total injection volume (1-3 ml) can be injected from 15 to 5 seconds into the vascular system of mice. In another embodiment the total injection volume (6-35 ml) can be injected into the vascular system of rats from 20 to 7 seconds. In another embodiment the total injection volume (80-200 ml) can be injected into the vascular system of monkeys from 120 seconds or less.

In another embodiment a large injection volume is used and the rate of injection is varied. Injection rates of less than 0.012 ml per gram (animal weight) per second are used in this embodiment. In another embodiment injection rates of less than 0.2 ml per gram (target tissue weight) per second are used for gene delivery to target organs. In another embodiment injection rates of less than 0.06 ml per gram (target tissue weight) per second are used for gene delivery into limb muscle and other muscles of primates.

Vessels comprise internal hollow tubular structures connected to a tissue or organ within the body of an animal, including a mammal. Bodily fluid flows to or from the body part within the lumen of the tubular structure. Examples of bodily fluid include blood, lymphatic fluid, or bile. Vessels comprise: arteries, arterioles, capillaries, venules, sinusoids, veins, lymphatics, and bile ducts. Afferent vessels are directed towards the organ or tissue and in which fluid flows towards the organ or tissue under normal physiological conditions. Conversely, efferent vessels are directed away from the organ or tissue and in which fluid flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. A vascular network consists of the directly connecting vessels supplying and/or draining fluid in a target organ or tissue.

The delivery process is effective in mice, rats, dogs, pig, and non-human primates. That delivery is observed in each of these animals strongly suggests that the processes are generally applicable to all mammals. In particular, the effectiveness of the processes in delivering molecules and complexes to nonhuman primates indicates that the processes will also be successful in humans.

The described processes may be combined with other delivery vehicles or vectors or other delivery enhancing groups. Such delivery vehicles and groups comprise: transfection reagents, viral vectors, non-viral vectors, lipids, polymers, polycations, amphipathic compounds, targeting signals, nuclear targeting signals, and membrane active compounds.

### EXAMPLES

1. Co-delivery of siRNA and morpholino antisense oligonucleotide to mammalian HeLa cells. HeLa cells were maintained in Dulbecco's Modified Eagle Medium supplemented with 10% fetal bovine serum. All cultures were maintained in a humidified atmosphere containing 5% CO₂ at 37° C. Approximately 24 h prior to transfection, cells were plated at an appropriate density in a T75 flask and incubated overnight. At 50% confluency, cells were initially transfected with pGL3 control (firefly luciferase, Promega, Madison Wis.) and pRL-SV40 (sea pansy luciferase, Promega, Madison, Wis.) using TransIT-LT1 transfection reagent according to the manufacturer's recommendations (Mirus Corporation, Madison, Wis.). 15 μg pGL3 control and 50 ng pRL-SV40 were added to 45 μl TransIT-LTI in 500 μl Opti-MEM (Invitrogen) and incubated 5 min at RT. DNA complexes were then added to cells in the T75 flask and incubated 2 h at 37° C. Cells were washed with PBS, harvested with trypsin/EDTA, suspended in media, plated into a 24-well plate with 250 μl DMEM+10% serum and incubated 2 h at 37° C. After incubation for 2 h, 400 μl DMEM/10% FBS was added to each well followed by the addition of siRNA complexed with TransIT-TKO (Mirus Corporation). For preparation of the siRNA and morpholino-containing complexes, 2 μl TransIT-TKO was diluted in 50 μl serum-free Opti-MEM and incubated at room temperature for 5 min. siRNA was added in order to give a final concentration of siRNA per well of 0, 0.1, or 10 nM and morpholino added to give a final concentration of morpholino per well of 0, 10, 100 or 1000 nM and incubated for 5 min at RT. Complexes were then added directly to the wells. All assay points were performed in duplicate wells. The pGL3 control plasmid contains the firefly luc+ coding region under transcriptional control of the simian virus 40 enhancer and early promoter region. The pRL-SV40 plasmid contains the coding region for Renilla reniformis, sea pansy, luciferase under transcriptional control of the simian virus 40 enhancer and early promoter region.

Morpholino antisense molecule and siRNAs used in this example were as follows:

Morpholino-Luc (GeneTools Philomath, Oreg.),

SEQ ID 1: 5′-TTATGTTTTTGGCGTCTTCCATGGT-3′(Luc+−3 to +22 of pGL3 Control Vector), was designed to base pair to the region surrounding the Luc+ start codon in order to inhibit translation of mRNA. Sequence of the start codon in the antisense orientation is underlined.

Standard control morpholino, SEQ ID 2: 5′-CCTCTTACCTCAGTTACAATTTATA-3′, contains no significant sequence identity to Luc+ sequence or other sequences in pGL3 Control Vector

GL3 siRNA-Luc+ (nucleotides 155-173 of Luc+ coding sequence):

SEQ ID 3

5′-rCrUrUrArCrGrCrUrGrArGrUrArCrUrUrCrGrAdTdT-3′:

SEQ ID 4

3′-dTdTrGrArArUrGrCrGrArCrUrCrArUrGrArArGrCrU-5′:

Single-stranded, gene-specific sense and antisense RNA oligomers with overhanging 3′ deoxynucleotides were prepared and purified by PAGE (Dharmacon, LaFayette, Colo.). The two complementary oligonucleotides, 40 μM each, are annealed in 250 μl 100 mM NaCl/50 mM Tris-HCl, pH 8.0 buffer by heating to 94° C. for 2 minutes, cooling to 90° C. for 1 minute, then cooling to 20° C. at a rate of 1° C. per minute. The resulting siRNA was stored at −20° C. prior to use.

In order to deliver the morpholino to cells in culture using the cationic transfection reagent, TransIT-TKO (Mirus Corporation) the morpholino was first annealed to a DNA oligonucleotide of complementary sequence. The sequence of the DNA strand is as follows: SEQ ID 5: 5′-GCCAAAAACATAAACCATGGAAGACT-3′. The morpholino and complementary DNA oligonucleotide, 0.5 mM each, were annealed in 5 mM Hepes, pH 8.0 buffer by heating to 94° C. for 2 minutes, cooling to 90° C. for 1 minute, then cooling to 20° C. at a rate of 1° C. per minute. The resulting morpholino/DNA complex was stored at −20° C. prior to use.

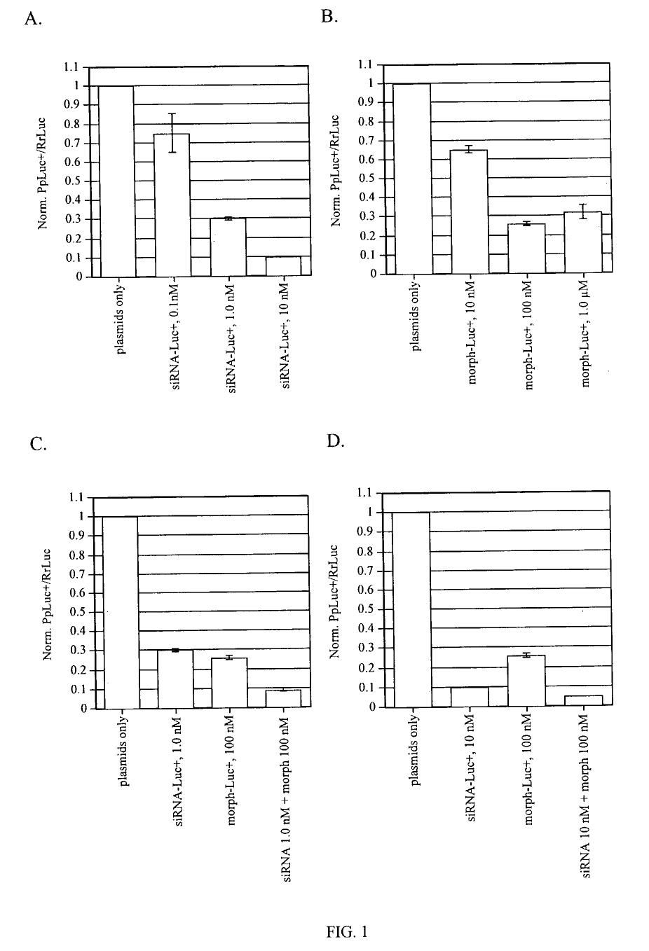

Cells were harvested after 24 h and assayed for luciferase activity using the Promega Dual Luciferase Kit (Promega). A Lumat LB 9507 (EG&G Berthold, Bad-Wildbad, Germany) luminometer was used. The amount of luciferase expression was recorded in relative light units. Numbers were then adjusted for control sea pansy luciferase expression and are expressed as the percentage of firefly luciferase expression in the absence of siRNA (FIG. 1) Numbers are the average for at least two separate wells of cells.

These data demonstrate that when siRNA and morpholino are delivery simultansously, the degree of inhibition is greater than with delivery of either siRNA or morphlino alone. For delivery of siRNA alone, a maximal inhibition of gene expression of 90% was observed for 10 nM siRNA. Maximal inhibition of gene expression observed for antisense morpholino alone was 75%. Delivery of 1.0 nM siRNA or 100 nM morpholino resulted in expressin inhibition of 70% and 75%, respectively. Co-delivery of 1.0 nM siRNA and 100 nM antisense morpholino resulted in greater than 90% inhibition. Co-delivery of 10 nM siRNA and 100 nM antisense morpholino resulted in greater than 95% inhibitionof luciferase expression.

2. Inhibition of Luciferase expression by delivery of antisense morpholino and siRNA simultaneously to liver in vivo. Morpholino antisense molecule and siRNAs used in this example were as follows:

DL94 morpholino (GeneTools Philomath, Oreg.), SEQ ID 1:

5′-TTATGTTTTTGGCGTCTTCCATGGT-3′(Luc+−3 to +22 of pGL3 Control Vector), was designed to base pair to the region surrounding the Luc+ start codon in order to inhibit translation of mRNA. Sequence of the start codon in the antisense orientation is underlined.

Standard control morpholino, SEQ ID 2: 5′-CCTCTTACCTCAGTTACAATTTATA-3′, contains no significant sequence identity to Luc+ sequence or other sequences in pGL3 Control Vector

GL3 siRNA-Luc+ (nucleotides 155-173 of Luc+ coding sequence):

SEQ ID 3: 5′-rCrUrUrArCrGrCrUrGrArGrUrArCrUrUrCrGrAdTdT-3′

SEQ ID 4: 3′-dTdTrGrArArUrGrCrGrArCrUrCrArUrGrArArGrCrU-5′

DL88:DL88C siRNA (targets nucleotides 765-783 of EGFP, GenBank#U76561):

SEQ ID 6

5′-rGrArArCrGrGrCrArUrCrArArGrGrUrGrArArCdTdT-3′:

SEQ ID 7

3′-dTdTrCrUrUrGrCrCrCrUrArGrUrUrCrCrArCrUrUrG-5′:

Two plasmid DNAs ±siRNA and ±antisense morpholino in 1-3 ml Ringer's solution (147 mM NaCl, 4 mM KCl, 1.13 mM CaCl₂) were injected, in 7-120 seconds, into the tail vein of mice. The plasmids were pGL3 control, containing the luc+ coding region under transcriptional control of the simian virus 40 enhancer and early promoter region, and pRL-SV40, containing the coding region for the Renilla reniformis luciferase under transcriptional control of the Simian virus 40 enhancer and early promoter region. 2 μg pGL3 control and 0.2 μg pRL-SV40 were injected with or without 5.0 μg siRNA and with or without 50 μg DL94 morpholino. One day after injection, the livers were harvested and homogenized in lysis buffer (0.1% Triton X-100, 0.1M K-phosphate, 1 mM DTT, pH 7.8). Insoluble material were cleared by centrifugation. The homogenate was diluted 10-fold in lysis buffer and 5 μl was assayed for Luc+ and Renilla luciferase activities using the Dual Luciferase Reporter Assay System (Promega Corp.). Ratios of Luc+ to Renilla Luc were normalized to the 0 μg siRNA-Luc+ control.

TABLE 1
Inhibition of luciferase expression from pGL3 control plasmid in mouse
liver after delivery of 50 μg antisense morpholino, 5 μg siRNA or both.

| | | percent inhibition of |
|---|---|---|
| Antisense morpholino | siRNA | luciferase expression |
| — | — | 0 |
| Standard | DL88:DL88C | 0 |
| DL94 | DL88:DL88C | 85.4 ± 2.7 |
| Standard | GL3 siRNA-Luc+ | 92.0 ± 1.9 |
| DL94 | GL3 siRNA-Luc+ | 98.6 ± 0.5 |

These experiments demonstrate the near complete inhibition of gene expression in vivo when antisense morpholino is delivered together with siRNA. This level of inhibition was greater than that for either morpholino or siRNA individually.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

References

1. Akhtar S, Hughes M D, Khan A, Bibby M, Hussain M, Nawaz Q, Double J, Sayyed P. The delivery of antisense therapeutics. Adv Drug Deliv Rev 2000; 44(l):3-21.

2. Boulikas, Teni, “Gene Therapy to Human Diseases: Ex vivo and In vivo Studies (Review).” International Journal of Oncology; 1996; vol. 9; pp. 1239-1251.

3. Budker V, Zhang G, Danko I, Williams P, Wolff J A. The Efficient Expression of Intravascularly Delivered DNA in Rat Muscle. Gene Therapy. 1998; 5: 272-276.

4. Caplen N J, Parrish S, Imani F, Fire A, Morgan R A. Specific inhibition of gene expression by small double-stranded RNAs in invertebrate and vertebrate systems. Proc Natl Acad Sci U S A 98:9742-9747, 2001.

5. Elbashir S M, Harborth J, Lendeckel W, Yalcin A, Weber K, Tuschl T. Duplexes of 21-nucleotide RNAs mediate RNA interference in cultured mammalian cells. Nature 2001; 411(6836):494-498.

6. French, Brent A., Et al., “Cellular and Molecular Cardiology: Percutaneous Transluminal In vivo Gene Transfer by Recombinant Adenovirus in Normal Porcine Coronary Arteries, Atherosclerotic Arteries, and Two Models of Coronary Restenosis.” Circulation; November 1994; vol. 90(5); pp. 2402-2413.

7. Garcia-Chaumont C, Seksek O, Grzybowska J, Borowski E, Bolard J. Delivery systems for antisense oligonucleotides. Pharmacol Ther. 2000; 87(2-3):255-277.

8. Hammond S M, Bernstein E, Beach D, Hannon G J. An RNA-directed nuclease mediates post-transcriptional gene silencing in Drosophila cells. Nature 404:293-296, 2000.

9. Liu Y, Liggitt D, Zhong W, Tu G, Gaensler K, Debs R. Cationic Liposome-Mediated Intravenous Gene Delivery. J Biol Chem 1995; 270(42): 24864-24870.

10. Liu F, Song Y K, Liu D. Hydrodynamics-based transfection in animals by systemic administration of plasmid DNA. Gene Therapy 1999; 6: 1258-1266.

11. Ogris M, Brunner S, Schuller S, Kircheis R, Wagner E. PEGylated DNA/Transferrin-PEI Complexes: Reduced Interaction with Blood Components, Extended Circulation in Blood and Potential for Systemic Gene Delivery. Gene Therapy 1999; 6:595-605.

12. Parrish S, Fleenor J, Xu S, Mello C, Fire A. Functional anatomy of a dsRNA trigger. Differential requirement for the two trigger strands in RNA interference. Mol Cell 6:1077-1087, 2000.

13. Rekhter, Mark D. MD, Phd, Et al., “Graft Penneabilization Facilitates Gene Therapy of Transplant Arteriosclerosis in a Rabbit Model.” Circulation; Sep. 29, 1998; vol. 98(13); pp. 1335-1341.

14. Rekhter, Mark D., Et al., “Gene Transfer Into Normal and Atherosclerotic Human Blood Vessels.” Circ. Res.; 1998; vol. 82; pp. 1243-1252.

15. Ross, Gail, Et al., “Gene Therapy in the United States: A Five-Year Status Report.” Human Gene Therapy; Sep. 10, 1996; vol. 7; pp. 1781-1790.

16. Sharp P A. RNA interference—2001. Genes Dev 2001; 15:485-490.

17. Simari, Robert D., Et al., “Regulation of Cellular Proliferation and Intimal Formation Following Balloon Injury in Atherosclerotic Rabbit Arteries.” Gene Therapy for Atherosclerotic Arteries; J. Clin. Invest.; July 1996; vol. 98, no. 1; pp. 225-235.

18. Tanner N. Ribozymes: the characteristics and properties of catalytic RNAs. FEMS Microbiology Reviews. 1999; 23(3):257-275.

19. Tuschl T, Zamore, P D, Lehmann R, Bartel D P, Sharp P A. Targeted mRNA degradation by double-stranded RNA in vitro. Genes Dev 1999; 13, 3191-3197.

20. Von Der Leyen, Heiko, E., Et al., “A Pressure-Mediatated Nonviral Method For Efficient Arterial Gene and Oligonucleotide Transfer.” Human Gene Therapy; Sep. 20, 1999; vol. 10; pp. 2355-2364.

21. Yu J Y, DeRuiter S L, Turner D L. RNA interference by expression of short-interfering RNAs and hairpin RNAs in mammalian cells. Proc Natl Acad Sci U S A 99(9): 6047-6052, 2002.

22. Zhang, Guofeng, Et al. Expression of Naked Plasmid DNA Injected into the Afferent and Efferent Vessels of Rodent and Dog Livers. Human Gene Therapy; Oct. 10, 1997; vol. 8; pp. 1763-1772.

23. Zhang G, Budker V, Williams P Subbotin V Wolff JA. Efficient Expression of Naked DNA Delivered Intraarterially to Limb Muscles of Nonhuman Primates. Hum Gene Ther. 2001; 12: 427-438.

24. Zhu N, Liggitt D, Liu Y, Debs R. Systemic Gene Expression After Intravenous DNA Delivery Into Adult Mice. Science 1993; 261:209-211.

25. PCT WO 03/010180 Kay M, McCaffrey A.; filed Feb. 6, 2003.

We claim:

1. A process for inhibiting gene expression in a cell comprising: delivering to the cell a combination of two or more RNA function inhibitors specific for the gene.

2. The process of claim 1 wherein at least one of the RNA function inhibitors induces RNA interference.

3. The process of claim 1 wherein at least one of the RNA function inhibitors consists of siRNA.

4. The process of claim 1 wherein one of the inhibitors consists of antisense polynucleotide.

5. The process of claim 3 wherein the antisense polynucleotide consists of a morpholino.

6. The process of claim 1 wherein at least one of the RNA function inhibitors is transcribed within the cell from a DNA expression cassette that is delivered to the cell.

7. The process of claim 6 wherein the expression cassette encodes an siRNA.

8. The process of claim 6 wherein the expression cassette encodes an antisense sequence.

9. The process of claim 1 wherein the combination of inhibitors consist of siRNA and antisense polynucleotide.

10. The process of claim 1 wherein the cell consist of an in vitro mammalian cell.

11. The process of claim 1 wherein the cell consists of an in vivo mammalian cell.

12. The process of claim 1 wherein inhibiting gene expression consists of providing a therapeutic effect.

13. The process of claim 12 wherein the gene consists of an infectious agent gene.

14. The process of claim 12 wherein the gene contributes to a disease state.

15. An in vivo process for reducing expression of a gene in a mammalian cell comprising:
a) inserting a combination of at least two RNA function inhibitors into the lumen of a vessel, b) increasing permeability of the vessel; and, c) delivering the inhibitors to an extravascular cell outside of the vessel via the increased permeability and reducing expression of the gene.

16. The process of claim 15 wherein at least one of the inhibitors consists of siRNA.

17. The process of claim 15 wherein at least one of the inhibitors consists of antisense polynucleotide.

18. The process of claim 15 wherein the inhibitors consist of siRNA and antisense polynucleotide.


SEQUENCE LISTING (7 sequences)

SEQ ID NO: 1 - DNA, 25 bp, Photinus pyralis
ttatgtttttggcgtcttccatggt

SEQ ID NO: 2 - DNA, 25 bp, Homo sapiens
cctcttacctcagttacaatttata

SEQ ID NO: 3 - DNA, 21 bp, Photinus pyralis
cuuacgcugaguacuucgatt

SEQ ID NO: 4 - DNA, 21 bp, Photinus pyralis
ttgaaugcgacucaugaagcu

SEQ ID NO: 5 - DNA, 26 bp, Photinus pyralis
gccaaaaacataaaccatggaagact

SEQ ID NO: 6 - DNA, 21 bp, Aequorea victoria
gaacggcaucaaggugaactt

SEQ ID NO: 7 - DNA, 21 bp, Aequorea victoria
ttcuugcccuaguuccacuug